(19) United States
(12) Patent Application Publication
(10) Pub. No.: US 2003/0050280 A1
(43) Pub. Date: Mar. 13, 2003

(54) Starch compositions containing biodegradation inhibitors and methods for the prevention of starch biodegradation

(75) Inventors: Philip Gerdon Sweeny, Hackettstown, NJ (US); Olga Borokhov, Chatham, NJ (US); Patrick Jay Lutz, Nazareth, PA (US); Ahmed Mohammed Tafesh, Flemington, NJ (US)

Correspondence Address: DARBY & DARBY P.C., P. O. BOX 5257, NEW YORK, NY, 10150-5257, US

(73) Assignee: LONZA INC.

(21) Appl. No.: 10/183,690

(22) Filed: Jun. 25, 2002

Related U.S. Application Data
(60) Provisional application No. 60/301,773, filed on Jun. 28, 2001.

(51) Int. Cl. A61K 31/718; A61K 31/4166

(52) U.S. Cl. 514/60; 514/390

(57) ABSTRACT

The present invention relates to a starch compositions that include biodegradation inhibitors which are low free formaldehyde formulation of one or more formaldehyde donor compounds. The starch compositions may also contain an additional inhibitor comprising one or more isothiazolone compounds which, when combined with the formaldehyde donor compounds, results in a synergistic blend. Methods for inhibiting starch biodegradation are also disclosed.

This application claims the benefit of U.S. Provisional Application No. 60/301,773, filed Jun. 28, 2001, which is hereby incorporated by reference.

### FIELD OF INVENTION

The present invention pertains to starch compositions containing biodegradation inhibitors and methods for preventing biodegradation of starch compositions.

### BACKGROUND OF THE INVENTION

The need for effective and economical means to preserve compositions prone to microbial attack is well known. In many industrial scenarios, there are a wide variety of applications where inhibiting the growth of microorganisms is necessary. Antimicrobial agents are useful in the production or use of paint, wood, textiles, adhesives, sealants, leather, rope, paper pulp, plastics, fuel, oil, and rubber and metal working fluids. Moreover, the control of slime-producing bacteria and fungi in pulp and paper mills and in cooling towers is a matter of substantial commercial importance.

The preservation of industrial starch slurries and pastes from the growth of microorganisms is of particular interest because industrial starch slurries and pastes are utilized in a variety of industrial processes where maintenance of their physical and chemical properties is required. For example, starch slurries and pastes are utilized in the paper industry as dry strength agents, surface sizes and coating binders. Inhibiting the growth of microorganisms in this environment is necessary to maintain low odor, a constant pH, viscosity, color, and degree of polymerization of the starch slurries and pastes since these characteristics impact the quality and performance properties of the produced papers.

Starch slurries and pastes consist of complex carbohydrates dispersed in an aqueous medium which provide an easily assimilated food source for bacteria, yeast and fungi. Consequently, starch slurries and pastes are regularly subjected to microbial contamination, even at elevated temperatures. Thermophilic, acid producing microorganisms are particularly troublesome because their metabolic processes cause biodegradation of the starch slurries and pastes. These microorganisms also remain viable at elevated temperatures, such as 150-160° F.

Thus, in order to preserve starch slurries and pastes from biodegradation, a preservative program is required. Due the commodity nature of many starch applications, it is necessary that such programs be highly cost-effective. In addition, as many antimicrobial programs are exceedingly toxic, it is desirable that such preservation programs offer minimized exposure risks to production personnel. This is especially true as elevated paste operational temperatures of 150-210° F. are frequently employed.

In light of the foregoing, it is clearly desirable to obtain starch preservation compositions which control microbiological and fungal contamination in starch products, such as starch slurries and pastes, at elevated temperatures, and that are highly cost-effective and provide minimized exposure risk profiles.

Certain compounds have long been known to be useful as preservatives. Compounds such as the halopropynyl carbamates are known for their fungicidal activity. However, they are costly and, as a result, have only found applications in specialty areas where the high costs can be justified.

Other commercially known preservatives include Quaternium-15 (Dowicil 200, a trademark of Dow Chemical Company). It has the disadvantage of being a solid product which must be solubilized in water before it can be used in the end product. In aqueous solution it exhibits pH drift and causes formulation problems, particularly with regard to viscosity and color.

Formaldehyde in the free state, as in formalin, is effective only for short periods of time and it is inactivated by protein. In addition, it is unacceptable from a toxicity and environmental viewpoint.

Alkyl parabens (e.g., methyl, ethyl, and propyl), which are useful as fungicides, have limited bactericidal action. They are generally solubilized in oil since they are poorly soluble in water, leading to formulation difficulties for personal care and household products. They are often inactivated by commonly used materials such as gelatin, methyl cellulose, and polyethylene glycol.

More recently, less toxic substances have been used as preservatives, including iodopropynyl butylcarbamate, polyaminopropyl biguanide, bis(3-aminopropyl) dodecylamine, benzethonium chloride, methyldibromo glutaronitrile, and ethylenediaminetetraacetic acid.

However, to obtain full microbiological control, a greater amount of these preservatives must be added to the product, thereby making it more difficult to formulate. Also, when large amounts of additive are used, the likelihood of a negative impact on that product, such as instability, odor and breakdown of product, is greater. Moreover, some of these compounds, such as iodopropynyl butylcarbamate, are costly, so the use of large amounts of these compounds is not economical.

These preservatives have also included formaldehyde and isothiazolinone derivatives. U.S. Pat. No. 3,987,184 shows the use of 1,3-dimethylol-5,5-dimethylhydantoin (DMDMH) as a useful formaldehyde donor compound for the preservation of personal care products, cosmetics, and household and industrial products. Mixtures of 5-chloro-2-methyl-3-isothiazolin-4-one (CMI) and 2-methyl-3-isothiazolin-4-one (MI) have also been used to preserve personal care, household, and industrial products.

The prior art also discloses the combination of a formaldehyde donor and one or more isothiazolone for the preservation of personal care, household, and industrial products. For example, a preservative system for clinical chemistry reagents comprising DMDMH and CMI/MI is disclosed by Voo et al. in U.S. Pat. No. 5,464,850. U.S. Pat. No. 6,114,366 to Lutz et al. discloses the use of a synergistic mixture of a formaldehyde donor and one or more isothiazolone in compositions that are at least 20% solids. U.S. Pat. No. 6,121,302 to Rothenberger et al. also discloses a preservative formulation including a formaldehyde donor and an isothiazolone. U.S. Pat. No. 6,133,300 to Smith et al. discloses the combination of 5,5-dimethylhydantoin (DMH) and 1,2 benbenzisothiazoin-3-one (BIT). Finally, EP 1084 619 discloses a stable composition of at least one imidazoldine, at least one 3-isothiazolone, a stabilizing amount of copper and a solvent. The above references are incorporated herein in their entirety.

These patents, however, do not disclose the present invention which involves starch compositions comprising novel low free formaldehyde formulations of formaldehyde donor compounds and starch compositions comprising the aforementioned formulations of low free formaldehyde donor compounds and the synergistic combination with one or more isothiazolones. These starch compositions are not susceptible to biodegradation, particularly at elevated temperatures.

### SUMMARY OF THE INVENTION

It has now been discovered that novel low free formaldehyde formulations of one or more formaldehyde donors may be used in the preservation of starch compositions. Such formulations are especially beneficial in the high operating temperatures of starch paste preparation and storage. This combination preferably includes a mixture of 1,3-dimethylol-5,5-dimethylhydantoin (DMDMH), 1-methylol-5,5-dimethylhydantoin/3-methylol-5,5-dimethylhydantoin (MMDMH) and 5,5-dimethylhydantoin (DMH).

It has also been surprisingly and unexpectedly discovered that when a second component comprising one or more isothiazolone compounds is added to one or more of the formaldehyde donor compounds, a synergistic effect regarding starch preservation occurs. Preferably, the isothiazolones are selected from the group consisting of 2-methyl-4-isothiazolin-3-one (MI), 5-chloro-2-methyl-4-isothiazolin-3-one (CMI), 1,2-benbenzisothiazoin-3-one (BIT) and mixtures thereof.

### DETAILED DESCRIPTION OF THE INVENTION

The present invention provides starch compositions comprising biodegradation inhibitors and methods of preserving starch compositions. The starch compositions of present invention include but are not limited to pastes and slurries.

The starch biodegradation inhibitors of the present invention comprise low free formaldyhyde formulations of one or more formaldehyde donors compounds, such as alkanoldialkyl hydantoins having the formula:

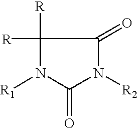

wherein each R is independently hydrogen, a methyl group, an ethyl group, a propyl group, an alkyl group or an aryl group, and R₁ and R₂ are each independently hydrogen or (CH₂)OH, with the proviso that both R₁ and R₂ cannot be hydrogen (i.e., at least one of R₁ and R₂ is (CH₂)OH)), and where the compound has chemical and physical characteristics compatible with use in starch compositions. The low free formaldehyde formulation may also include a dialkyl hydantoin having the formula

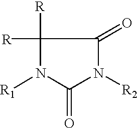

where R is as defined above and R₁ and R₂ are hydrogen. The mixture preferably includes 1,3-dimethylol-5,5-dimethylhydantoin (DMDMH), 1-methylol-5,5-dimethylhydantoin/3-methylol-5,5-dimethylhydantoin (MMDMH) and 5,5-dimethylhydantoin (DMH).

The preferred concentration of free formaldehyde in the low free formaldehyde formulation is less than 0.2% by weight based on 100% weight of the starch composition. More preferably, the concentration of free formaldehyde in the low free formaldehyde formulation is less than 0.1% by weight based on 100% weight of the starch composition.

The starch compositions of the present invention may include a second component in addition to the formaldehyde donor compounds discussed above. The second component comprises one or more isothiazolones of the following formulas:

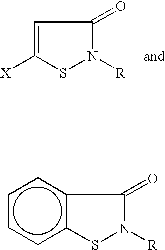

wherein X is hydrogen or halogen, preferably chlorine, and R is a hydrogen, an alkyl chain of from 1 to 22 carbon atoms, a cycloalkyl group of 3 to 8 carbon atoms, an aralkyl group of up to 8 carbon atoms, an aryl or substituted aryl group of 6 carbon atoms, a benzyl group, a halogen, C₁-C₄ alkyl or C₁-C₄ alkoxy-substituted benzyl group, a carbalkoxyalkyl group of up to 12 carbon atoms, a dialkylaminoalkyl group of up to 12 carbon atoms, a haloalkyl group of up to 12 carbon atoms, an alkoxyalkyl group of up to 12 carbons atoms, an alkylthioalkyl group of up to 12 carbon atoms, an alkenyl group of up to 12 carbon atoms, an alkynyl group of up to 12 carbon atoms, an alkali metal ion or an alkaline earth ion such as Na, Li, K, Ca or Mg. It is understood that commercial samples of isothiazolones contain stabilizers known in the art and the presence of these stabilizers are incorporated by reference.

The isothiazolone compounds are preferably selected from the group consisting of 2-methyl-4-isothiazolin-3-one (MI), 5-chloro-2-methyl-4-isothiazolin-3-one (CMI), 1,2-benzisothiazoin-3-one (BIT) and alkali metal salts of BIT and mixtures thereof. The isothiazolone compounds are typically obtained by diluting an aqueous stock solution that contains one or more isothiazolones of the above formula, including stock solutions containing isothiazolone stabilizers known in the art.

Preferably, the inhibitors are added to the starch slurries or pastes in amounts such that the ratio of the first component (formaldehyde donor) to the second component (isothiazolone) ranges from about 1:1 to about 10,000:1. More preferably, the ratio of the first component to the second component ranges from about 200:1 to about 500:1.

According to one embodiment, the concentration of the first component of the system ranges from about 50 ppm to about 5000 ppm. A preferred range for the concentration of the first component of the system ranges from about 50 ppm to about 1000 ppm. A more preferred range for the first component ranges from about 100 ppm to about 500 ppm.

A preferred range for the concentration of the second component ranges from about 0.05 ppm to about 100 ppm. A more preferred concentration for the second component ranges from about 0.1 ppm to about 50 ppm.

It is a surprising and unexpected finding of this invention that bound formaldehyde formulations of DMH, DMDMH and MMDMH providing low free formaldehyde residuals may be effectively used to prevent biodegradation of starch compositions. To this formulation, CMI, MI and BIT, and mixtures thereof may be added thereby producing a synergistic result.

Specifically, solutions of 5,5-dimethylhydantoin (DMH) such as Dantogard® (Lonza Inc. Fairlawn N.J.), Dantogard® 2000 (Lonza Inc. Fairlawn N.J.), and blends of DMDMH, MMDMH and/or DMH with MI/CMI and/or BIT such as Lonzaserve® SG (Lonza Inc., Fairlawn N.J.) may be used to effectively reduce microbial populations and maintain physical-chemical properties of industrial starch slurries and pastes.

The starch biodegradation inhibitors of the present invention inhibit the production of acids from microorganisms present in the starch compositions. Such microorganisms, if not controlled, cause precipitous pH depression accompanied by increased hydrolysis, loss of viscosity and other important physical-chemical properties of the industrial starch compositions. In addition to inhibition of acid production of the microorganisms, the present invention inhibits the depolymerization of the starch compositions by microbe produced enzymes, such as amylase.

The present invention is directed to starch compositions containing the formulations and synergistic blends described above. The definition of a synergistic effect is a response to a combination of two or more components that is greater than the sum of its parts. A mathematical approach for measuring synergy using the Kull synergy index (Kull et al. Applied Microbiology 1961, 9, 538-541) was performed using the following relationship:

$$Synergism Index ( SI ) = \frac{{Q}_{A}}{{Q}_{a}} + \frac{{Q}_{B}}{{Q}_{b}}$$

Where:

Qₐ=The quantity of Compound a acting alone, producing an endpoint.

Q_{b}=The quantity of Compound b acting alone, producing an endpoint.

Q_{A}=The quantity of Compound A in mixture, producing an endpoint.

Q_{B}=The quantity of Compound B in mixture, producing an endpoint.

When SI is equal to 1, a mere additive effect of the components in the mixture is indicated; when SI is less than 1, synergism has occurred; and when SI is greater than 1, antagonism of the two components has occurred.

The following examples are illustrative of the present invention. However, it will be understood that the invention is not limited to the specific details set forth in the examples.

### EXAMPLE 1

Lonzaserve®, Dantogard® and Isocil® (a 1.5% CMI/Ml mixture, Lonza Inc. Fairlawn, N.J.) preservation efficacy was evaluated in a cooked dry strength starch (Redibond 2038 National Starch, Bridgewater, N.J.). Starch spoilage microorganisms were those which naturally proliferated in the aqueous dispersed sample. Contaminated starch samples containing about 3-4×10⁷ cfu/ml of bacteria were used to evaluate the preservative candidates. To sterile bottles, 40 g of starch dispersion were transferred. Preservative candidates were added to the samples at target concentrations. The test samples were sealed and stored at 20° C. for 28 days of contact time. The control sample contained no preservative.

The number of viable microorganisms present at 0, 7, 14, and 28 days were evaluated by standard pour plate techniques. One gram of the test samples was neutralized using D/E Neutralizing Broth and then serially diluted. Samples were plated on Tryptic Soy Agar. Plates were incubated for 48 hours at 37° C. for bacterial plate counts.

As shown in Table 1, the Lonzaserve® DMDMH/MI/CMI mixture provided 2 log reductions in 7 days at 284/0.0904 ppm active ingredients levels and 2 log reductions in 14 days at 142/0.452 ppm active ingredients relative to the untreated control. The Dantogard® DMDMH formulation provided 2 log reductions in 14 days at 340 and 170 ppm active ingredients. The Isocil® MI/CMI formulation did not achieve 2 log reductions at even the highest tested concentration.

In summary, both the DMDMH/Ml/CMI blend and the DMDMH formulation showed excellent and unexpected starch preservation efficacy.

TABLE 1
Efficacy results (house contaminate, cooked starch slurry).

| | Concentration, | Sample Bacterial Enumeration (cfu/ml) | | | | |
|---|---|---|---|---|---|---|
| | product | Challenge Days | | | | |
| | (ppm active) | 0 | 7 | 14 | 21 | 28 |
| DMDMH/ | 0.2% | 4.0 × 10⁷ | 9.0 × 10⁵ | 3.0 × 10³ | 4.8 × 10³ | 4.5 × 10³ |
| MI/CMI | 284/0.904 | | | | | |
| mixture | form./MI + CMI | | | | | |
| | 0.1% | 4.0 × 10⁷ | 4.8 × 10⁶ | 2.1 × 10⁴ | 1.4 × 10⁴ | 1.5 × 10⁴ |
| | 142/0.452 | | | | | |
| | 0.05% | 3.6 × 10⁷ | 6.1 × 10⁶ | 1.3 × 10⁶ | 3.2 × 10⁵ | 7.7 × 10⁴ |
| | 71/0.23 | | | | | |
| DMDMH | 0.2% | 3.9 × 10⁷ | 9.0 × 10⁵ | 6.0 × 10³ | 9.2 × 10³ | 3.1 × 10³ |
| | 220 | | | | | |
| | form. | | | | | |
| | 0.1% | 4.0 × 10⁷ | 5.0 × 10⁶ | 3.0 × 10⁴ | 5.5 × 10⁴ | 9.3 × 10³ |
| | 110 | | | | | |
| | 0.05% | 4.0 × 10⁷ | 5.5 × 10⁶ | 1.5 × 10⁶ | 6.3 × 10⁵ | 8.0 × 10⁴ |
| | 55 | | | | | |
| MI/CMI | 0.075% | 3.7 × 10⁷ | 8.0 × 10⁶ | 2.0 × 10⁶ | 1.5 × 10⁶ | 2.8 × 10⁵ |
| | 11 | | | | | |
| | MI + CMI | | | | | |
| | 0.05% | 4.0 × 10⁷ | 1.0 × 10⁷ | 1.5 × 10⁶ | 1.4 × 10⁶ | 5.1 × 10⁵ |
| | 6.5 | | | | | |
| Unpreserved | 0 | 3.7 × 10⁷ | 1.0 × 10⁷ | 4.2 × 10⁶ | 4.4 × 10⁶ | 1.9 × 10⁶ |
| Control | | | | | | |

### EXAMPLE 2

Lonzaserve® SG (DMDMH/MMDMH/DMH and MI/CMI blend), a DMDMH/MMDMH/DMH and BIT blend, Dantogard® 2000 (DMDMH/MMDMH/DMH) and Kathon® CG (a 1.5% CMI/MI mixture, Rohm and Haas, Philadelphia, Pa.) preservation efficacy was evaluated in an uncooked ethoxylated starch slurry (Ethylex 2025, Staley, Decatur Ill.). A fresh starch slurry solution containing 23% solids in sterile tap water was prepared on the date of experiment (pH=7.8, T=37° C.). The test preservatives were added to 70 ml of the starch slurry in 125-ml sterile capped Erlenmeyer flasks. No preservative was added to the untreated control.

A gram negative bacteria Pseudomonas aeruginosa ATCC 9027 was used for evaluation of the preservative candidates. The bacterial strain was maintained and grown on Tryptic Soy Agar. Inoculum was prepared by washing the surface of the 18-24 hours slants with phosphate buffer water (pH=7.2) in order to obtain a microbial count in the inoculum of 1-2×10⁹. Each sample was treated with the inoculum to achieve a count of 5×10⁶ cfu/ml of bacteria in the test samples. The unpreserved control contained no biocide. The test samples were incubated at 37° C. on an orbital shaker (80-100 rpm) for 4, and 24 hour of contact times. The number of viable microorganisms present in the each test sample were evaluated by standard pour plate techniques. One gram of these test samples was neutralized using D/E Neutralizing Broth and then serially diluted.samples were plated on Tryptic Soy Agar (bacteria). Plates were incubated for 48 hours at 37° C.

Results are reported as the percent reduction using the follow equation:

Reduction, %=(A−B)/A×100,

where:

A—count of microorganisms in the unpreserved sample

B—count of microorganisms in the test sample

A reduction in the level of microorganisms of more than 99.99% (4 Logs) compared to the unpreserved control was selected as the criteria of biocide effectiveness for the uncooked starch slurry test.

As shown in Table 2, the Lonzaserve® DMDMH/MMDMH/DMH and MI/CMI blend provided 5 log reductions in 4 hours at 142/0.452 ppm active ingredients relative to the untreated control. The Dantogard® 2000 DMDMH/MMDMH/DMH formulation provided 4 log reductions in 4 hours at 340 ppm active ingredients. The Kathon®) CG MI/CMI formulation did not achieve 4 log reductions in 4 hours at even the highest tested concentration of 11 ppm active ingredients. The DMDMH/MMDMH/DMH and BIT blend provided 6 log reductions in 4 hours at 284/21.6 ppm active ingredients.

In summary, the DMDMH/MMDMH/DMH and MI/CMI blend, the DMDMH/MMDMH/DMH and BIT blend and the DMDMH/MMDMH/DMH formulation showed excellent and unexpected starch preservation efficacy.

TABLE 2
Efficacy Results (P. aeruginosa, uncooked starch slurry).

| | | | | Microbial Efficacy, | | | | |
|---|---|---|---|---|---|---|---|---|
| | Concentration, | | | Log Reduction | | | pH | |
| | % | ppm | Inoculum, | 4 | 24 | 0 | 24 | 48 |
| Compound | product | active | Cfulml | hours | hours | hours | hours | hours |
| MI/CMI | 0.075 | 11 | 5 × 10⁶ | 3 | 4 | 7.8* | 6.72 | 6.63 |
| DMDMH | 0.05 | 85 | 5 × 10⁶ | 0 | 0 | 7.8* | 7.26 | 6.65 |
| | 0.1 | 170 | 5 × 10⁶ | 0 | 6 | 7.8* | 8.26 | 8.20 |
| | 0.2 | 340 | 5 × 10⁶ | 4 | 6 | 7.8* | 8.26 | 8.29 |
| | 0.3 | 510 | 5 × 10⁶ | 5 | 6 | 7.8* | 8.22 | 8.15 |
| DMDMH/MI/CMI | 0.05 | 71/0.23 | 5 × 10⁶ | 0 | 0 | 7.8* | 7.23 | 6.74 |
| | 0.1 | 142/0.452 | 5 × 10⁶ | 5 | 6 | 7.8* | 8.43 | 8.24 |
| | 0.2 | 284/0.904 | 5 × 10⁶ | 5 | 6 | 7.8* | 8.28 | 8.16 |
| DMDMH/BIT | 0.1 | 284/21.6 | 5 × 10⁶ | 6 | 6 | 7.8* | 8.28 | 8.28 |
| | 0.2 | 568/44.1 | 5 × 10⁶ | 6 | 6 | 7.8* | 8.14 | 8.08 |
| Unpreserved | — | — | 5 × 10⁶ | 0 | +1 | 7.8 | 6.81 | 6.43 |
| sample | | | | | | | | |

*initial pH was assumed to be equal to pH of blank sample.

### EXAMPLE 3

Synergistic efficacy was observed for the DMDMH/MMDMH/DMH and MI/CMI blend and is expected for the DMDMH/MMDMH/DMH and BIT blend. Analysis of the microbial efficacy results of Table 2 using the Kull synergy index (Kull et al. Applied Microbiology 1961, 9, 538-541) was performed using the following relationship:

$$Synergism Index ( SI ) = \frac{{Q}_{A}}{{Q}_{a}} + \frac{{Q}_{B}}{{Q}_{b}}$$

Where:

Qₐ=The quantity of Compound a acting alone, producing an endpoint.

Q_{b}=The quantity of Compound b acting alone, producing an endpoint.

Q_{A}=The quantity of Compound A in mixture, producing an endpoint.

Q_{B}=The quantity of Compound B in mixture, producing an endpoint.

When SI is equal to 1, a mere additive effect of the components in the mixture is indicated; when SI is less than 1, synergism has occurred; and when SI is greater than 1, antagonism of the two components has occurred. According to this well known method of measuring synergism, the quantity of each component in the various mixtures is compared with the quantity of pure component that is required to reach the same endpoint or to produce the same microbiological effect as the mixture. The results of this analysis are shown in Table 3.

As shown in Table 3, the DMDMH/MMDMH/DMH and MI/CMI blend produces a synergistic effect as demonstrated by the observed synergy index value of 0.44 for the antimicrobial results.

### EXAMPLE 4

Maintenance of starch solution pH is as important as bacterial population control. A pH drop over 24-48 hours is undesirable as it can affect the physical-chemical properties of the starch. Using the criteria of pH maintenance above 7.8 for 24 and 48 hours the DMDMH/MMDMH/DMH and MI/CMI blend is shown to be synergistic as indicated by the synergy index of 0.89 calculated in Table 3.

TABLE 3
Synergism index for efficacy and pH test results

| | | | | | Synergism Index (SI) | If SI ≦ 1 |
|---|---|---|---|---|---|---|
| Parameter | Q_{A} | Q_{B} | Qₐ | Q_{b} | Qₐ/Q_{A} + Q_{b}/Q_{B} = SI | Synergy |
| Efficacy | 340 ppm | 11 ppm > | 142 ppm | 0.452 ppm | 142/340 + 0.452/11 = 0.44 | <1 |
| | of DMDMH | of | of DMDMH in | of MI/CMI in | | synergy |
| | | MI/CMI | mixture | mixture | | |
| pH | 170 ppm | 11 ppm > of | 142 ppm | >0.452 ppm | 142/107 + 0.452/11 = 0.89 | <1 |
| | of DMDMH | MI/CMI | of DMDMH in | of MI/CMI in | | synergy |
| | | | mixture | mixture | | |

What is claimed is:

1. A starch composition comprising a biodegradation inhibitor which is a low free formaldehyde formulation of one or more formaldehyde donor compounds having the formula: 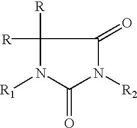
wherein each R is independently hydrogen, a methyl group, an ethyl group, a propyl group, or an aryl group; R₁ and R₂ are each independently hydrogen or (CH₂)OH; and at least one of R₁ and R₂ is (CH₂)OH.

2. The starch composition according to claim 1, wherein the low free formaldehyde formulation comprises formaldehyde donor compounds selected from the group consisting of 1,3-dimethylol-5,5-dimethylhydantoin (DMDMH), 1-methylol-5,5-dimethylhydantoin (MMDMH), 3-methylol-5,5-dimethylhydantoin (MMDMH), 5,5-dimethylhydantoin (DMH), and mixtures thereof.

3. A starch composition comprising a biodegradation inhibitor which is an antimicrobially synergistic combination of a first component and a second component,
the first component comprising an antimicrobial low free formaldehyde formulation of one or more formaldehyde donor compounds having the formula: 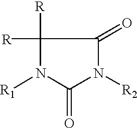
wherein each R is independently hydrogen, a methyl group, an ethyl group, a propyl group, or an aryl group; R₁ and R₂ are each independently hydrogen or (CH₂)OH; and at least one of R₁ and R₂ is (CH₂)OH; and
the second component comprising at least one isothiazolone having a formula selected from the group consisting of 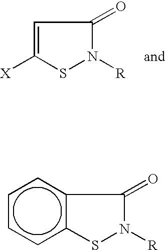
wherein X is hydrogen or halogen and R is a hydrogen, an alkyl chain of from 1 to 22 carbon atoms, a cycloalkyl group of 3 to 8 carbon atoms, an aralkyl group of up to 8 carbon atoms, an aryl or 16 substituted aryl group of 6 carbon atoms, a benzyl group, a halogen, C₁-C₄ alkyl- or C₁-C₄ alkoxy-substituted benzyl group, a carbalkoxyalkyl group of up to 12 carbon atoms, a dialkylaminoalkyl group of up to 12 carbon atoms, a haloalkyl group of up to 12 carbon atoms, an alkoxyalkyl group of up to 12 carbons atoms, an alkylthioalkyl group of up to 12 carbon atoms, an alkenyl group of up to 12 carbon atoms, an alkynyl group of up to 12 carbon atoms, an alkali metal ion or an alkaline earth ion.

4. The composition according to claim 3, wherein the isothiazolone contains a stabilizer.

5. The starch composition according to claim 3, wherein the isothiazolone is selected from the group consisting of 2-methyl-4-isothiazolin-3-one (MI), 5-chloro-2-methyl-4-isothiazolin-3-one (CMI), 1,2-benbenzisothiazoin-3-one (BIT), an alkali metal salt of BIT and mixtures thereof.

6. The composition according to claim 3, wherein the weight ratio of the first component to the second component ranges from about 1:1 to about 10,000:1.

7. The composition according to claim 3, wherein the weight ratio of the first component to the second component ranges from about 200:1 to about 500:1.

8. The composition according to claim 3, wherein the concentration of the first component ranges from about 50 ppm to about 5000 ppm.

9. The composition according to claim 3, wherein the concentration of the first component ranges from about 50 ppm to about 1000 ppm.

10. The composition according to claim 3, wherein the concentration of the first component ranges from about 100 ppm to about 500 ppm.

11. The composition according to claim 3, wherein the concentration of the second component ranges from about 0.05 ppm to about 100 ppm.

12. The composition according to claim 3, wherein the concentration of the second component ranges from about 0.1 ppm to about 50 ppm.

13. A method for preventing biodegradation of starch compositions which comprises contacting the starch composition with a biodegradation inhibitor which is an antimicrobial low free formaldehyde formulation of one or more formaldehyde donor compounds having the formula: 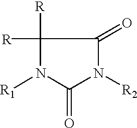
wherein each R is independently hydrogen, a methyl group, an ethyl group, a propyl group, an alkyl group or an aryl group; and R₁ and R₂ are each independently hydrogen or (CH₂)OH; and at least one of R₁ and R₂ is (CH₂)OH.

14. The method of claim 13, wherein the antimicrobially low free formaldehyde formulation comprises formaldehyde donor compounds selected from the group consisting of 1,3-dimethylol-5,5-dimethylhydantoin (DMDMH), 1-methylol-5,5-dimethylhydantoin (MMDMH), 3-methylol-5,5-dimethylhydantoin (MMDMH), 5,5-dimethylhydantoin (DMH), and mixtures thereof.

15. The method of claim 13, wherein the concentration of free formaldehyde in the low free formaldehyde formulation is less than 0.2% by weight based on 100% weight of the starch composition.

16. The method of claim 14, wherein the concentration of free formaldehyde in the low free formaldehyde formulation is less than 0.1% by weight based on 100% weight of the starch composition.

17. A method for preventing biodegradation of starch compositions which comprises contacting the starch composition with a biodegradation inhibitor which is an antimicrobially synergistic combination of a first component and a second component,
the first component comprising an antimicrobial low free formaldehyde formulation of one or more formaldehyde donor compounds having the formula: 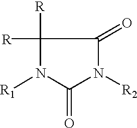
wherein each R is independently hydrogen, a methyl group, an ethyl group, a propyl group, or an aryl group; R₁ and R₂ are each independently hydrogen or (CH₂)OH; and at least one of R₁ and R₂ is (CH₂)OH; and the second component comprising at least one isothiazolone having a formula selected from the group consisting of: 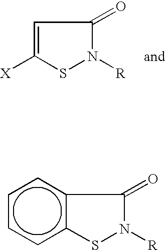
wherein X is hydrogen or halogen, preferably chlorine, and R is a hydrogen, an alkyl chain of from 1 to 22 carbon atoms, a cycloalkyl group of 3 to 8 carbon atoms, an aralkyl group of up to 8 carbon atoms, an aryl or substituted aryl group of 6 carbon atoms, a benzyl group, a halogen, C₁-C₄ alkyl- or C₁-C₄ alkoxy-substituted benzyl group, a carbalkoxyalkyl group of up to 12 carbon atoms, a dialkylaminoalkyl group of up to 12 carbon atoms, a haloalkyl group of up to 12 carbon atoms, an alkoxyalkyl group of up to 12 carbons atoms, an alkylthioalkyl group of up to 12 carbon atoms, an alkenyl group of up to 12 carbon atoms, an alkynyl group of up to carbon atoms, an alkali metal ion or an alkaline earth ion.

18. The method of claim 17, wherein the isothiazolone compound is selected from the group consisting of 2-methyl-4-isothiazolin-3-one (MI), 5-chloro-2-methyl-4-isothiazolin-3-one (CMI), 1,2-benbenzisothiazoin-3-one (BIT), an alkali metal salt of BIT, and mixtures thereof.

19. The method of claim 17, wherein the weight ratio of the first component to the second component ranges from about 1:1 to about 10,000:1.

20. The method of claim 17, wherein the weight ratio of the first component to the second component ranges from about 200:1 to about 500:1.

21. The method of claim 17, wherein the concentration of the first component ranges from about 50 ppm to about 5000 ppm.

22. The method of claim 17, wherein the concentration of the first component ranges from about 50 ppm to about 1000 ppm.

23. The method of claim 17, wherein the concentration of the first component ranges from about 100 to about 500 ppm.

24. The method of claim 17, wherein the concentration of the second component ranges from about 0.05 ppm to about 100 ppm.

25. The method of to claim 17, wherein the concentration of the second component ranges from about 0.1 ppm to about 50 ppm.
